(19) United States
(12) Patent Application Publication
(10) Pub. No.: US 2011/0301434 A1
(43) Pub. Date: Dec. 8, 2011

(54) Implantable Device and Surgical Implantation Technique

(76) Inventors: Razi-ul Haque, Ann Arbor, MI (US); Kensall Wise, Ann Arbor, MI (US); Paul R. Lichter, Ann Arbor, MI (US)

(21) Appl. No.: 13/154,291

(22) Filed: Jun. 6, 2011

Related U.S. Application Data
(60) Provisional application No. 61/351,741, filed on Jun. 4, 2010.

(51) Int. Cl. A61B 5/00

(52) U.S. Cl. 600/300

(57) ABSTRACT

An implantable device is provided, along with a surgical technique for implanting the implantable device. The implantable device includes a body, at least two fingers, and a diagnostic tool. The body presents a generally flat configuration. The at least two fingers extend from opposite sides of the body along a common plane with the flat configuration of the body. For the surgical technique, the implantable device is positioned above flexible and elastic tissue at a target location. The finger on one side of the body is slid under a portion of the flexible and elastic tissue without penetrating the flexible and elastic tissue. The finger on the opposing side of the body is slid under another portion of the flexible and elastic tissue without penetrating the flexible and elastic tissue. The flexible and elastic tissue exerts force perpendicular to the body and fingers to fixate the implantable device.

### CROSS-REFERENCE TO RELATED APPLICATIONS

This application claims priority to and all the advantages of U.S. Provisional patent application No. 61/351,741, filed on Jun. 4, 2010.

### GOVERNMENT LICENSE RIGHTS

This invention was made with government support under EEC9986866 awarded by National Science Foundation. The government has certain rights in the invention.

### BACKGROUND OF THE INVENTION

1. Field of the Invention

The present invention generally relates to an implantable device and a surgical technique for implanting the same. In particular, the present invention relates to an implantable device that can be fixated to flexible and elastic tissue without sutures, and a surgical technique for fixating the implantable device to flexible and elastic tissue without sutures.

2. Description of the Related Art

Implantable devices have various uses in diagnostics and monitoring applications within bodies of organisms. Such implantable devices are often required to be fixated to tissue within the body to prevent migration of the devices once implanted and to maintain the devices at desired locations for performing the desired diagnostics or monitoring. However, conventional methods of fixating implantable devices, such as through sutures, may result in tissue damage that causes discomfort and that may cause permanent damage. Such conventional methods of fixating implantable devices can be particularly problematic for sensitive tissues, such as tissues in the eye, where implantable devices would be useful.

Methods for fixating intraocular implantable devices to tissue that do not require sutures have been developed. For example, an intraocular implantable device has been developed that includes a protruding anchor extending from a planar surface thereof. The anchors of the previously-developed devices are configured and arranged to match the topology and features of the iris of the eye, which has folds that are capable of receiving the anchor and securing the implantable device in place. While the existing intraocular implantable devices minimize invasiveness and reduce tissue damage, the existing devices require features within the tissue to secure the devices. Such features within the tissue exhibit great variation from individual to individual and it may be difficult to identify suitable locations for securing the previously-developed devices in any particular individual.

In view of the foregoing, there remains an opportunity to further develop implantable devices and surgical implantation techniques that do not require use of sutures to minimize invasiveness and reduce tissue damage, but that also overcome the above problems with existing implantable devices and surgical implantation techniques.

### DESCRIPTION OF THE DRAWINGS

Other advantages of the present invention will be readily appreciated, as the same becomes better understood by reference to the following detailed description when considered in connection with the accompanying drawings wherein:

FIG. 1 is a photograph of implantable devices in accordance with the instant invention shown on a penny to illustrate the size of the devices;

FIGS. 2A-G are schematic top views of various finger configurations for implantable devices in accordance with the instant invention;

FIG. 3 is a schematic perspective view of a body of one embodiment of an implantable device in accordance with the instant invention showing an integrated circuit therein;

FIG. 4 is a schematic cross-sectional side view of an implantable device in accordance with the instant invention once implanted in flexible and elastic tissue such as an iris of an eye;

FIG. 5 is a photograph of an implantable device in accordance with the instant invention implanted in the iris of a cadaver eye; and

FIG. 6 is a photograph of an implantable device in accordance with the instant invention showing an integrated circuit embedded in the body thereof.

### SUMMARY OF THE INVENTION AND ADVANTAGES

The present invention provides an implantable device that can be fixated to flexible and elastic tissue without sutures and a surgical technique for implanting the implantable device. The implantable device includes a body, at least two fingers, and a diagnostic tool. The body presents a generally flat configuration. The at least two fingers extend from opposite sides of the body along a common plane with the flat configuration of the body. The surgical technique includes providing the implantable device. The implantable device is positioned above the flexible and elastic tissue at a target location. The finger on one side of the body is slid under a portion of the flexible and elastic tissue without penetrating the flexible and elastic tissue. The finger on the opposing side of the body is slid under another portion of the flexible and elastic tissue without penetrating the flexible and elastic tissue. Once the fingers on the opposing sides of the body are slide under the portions of the flexible and elastic tissue, the flexible and elastic tissue exerts force perpendicular to the body and fingers to fixate the implantable device.

The implantable devices of the instant invention have the advantage of being implantable through minimally invasive techniques, such as through the surgical technique in accordance with the instant invention, such that the need for sutures may be drastically minimized or eliminated when compared to existing devices and techniques. Even more, as implantable devices are reduced in size, it becomes increasingly challenging for skilled surgeons to accurately place sutures and, even then, the sutures may not provide sufficient force to secure the device due the reduced dimensions, thus further highlighting the benefits of the implantable devices of the instant invention. Furthermore, the device and technique of the instant invention overcome the problems with existing implantable devices and surgical implantation techniques because implantation does not require a particular topography of the tissue into which the device is implanted and can be implanted at virtually any location in tissue that is flexible and elastic.

### DETAILED DESCRIPTION OF THE INVENTION

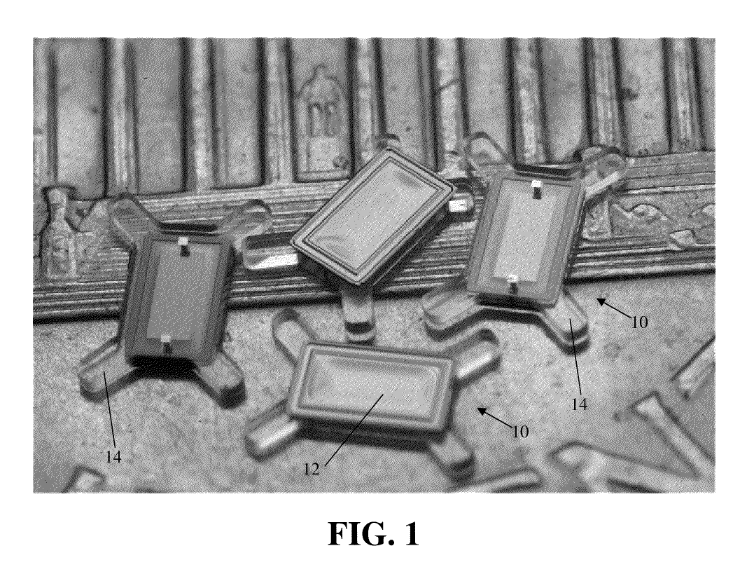

Referring to the Figures, where like numerals indicate like or corresponding parts through the several views, implantable devices are shown at 10 in FIGS. 1-3. Microfabrication technology, or Microelectromechanical Systems (MEMS), may be used to fabricate implantable devices 10 with dimensions on the order of millimeters or smaller (as illustrated with comparison to features of a penny in FIG. 1). By incorporating special microfabricated features on these devices on the sub-millimeter scale, these devices can be implanted surgically in new ways as well. The implantable devices 10 may be surgically implanted into organisms and fixated to flexible and elastic tissue without sutures. The implantable devices can be implanted in any flexible and elastic tissue that exhibits stretchiness, such as muscle, collagen, etc., and is held in place by exploiting compressive forces on the implantable device 10 that arise from a desire of the tissue to return to a normal state from a stretched state. For example, the tissue may be the iris of the eye, which is constructed of muscle and collagen and therefore provides both flexible and elastic properties.

The implantable devices 10 are designed specifically to aid surgical implantation. The implantable devices 10 include a body 12, at least two fingers 14, and a diagnostic tool. The body 12 presents a generally flat configuration. In particular, the body 12 is typically planar with a smaller thickness as compared to length or width. The body 12 typically has a length and width on the millimeter scale, i.e., a length and width of less than 1 cm. In one embodiment, the body 12 presents a length of from about 0.5 to about 2.5 mm, alternatively from about 1.0 to about 2.0 mm, and a width of from about 0.5 to about 2.5 mm, alternatively from about 1.0 to about 2.0 mm, alternatively from about 1.0 to 1.5 mm. In one particular example, the body 12 has a width/length of 1.2 mm×2 mm. Such dimensions generally enable implantation of the devices with minimal invasiveness. Particular for the embodiment in which the implantable device 10 is intended for implantation in the iris of the eye, the device 10 should be small enough to avoid occluding the field of view as the iris controls the dilation of the pupil to adjust the amount of light entering the eye. In low-light situations, the pupil might not be allowed to dilate enough if the device 10 is too large, partially occluding vision. The ranges of lengths and widths provided above for the device 10 may enable the device 10 to avoid scratching the endothelial cells of the cornea, which could damage them and affect vision. The body 12 is not limited to any particular shape, and the shape may be dictated by the requirements of the diagnostic tool that is included in the implantable device 10. Thus, although the implantable devices 10 are shown in the Figures to have a generally rectangular shape as defined by the length and width thereof, it is to be appreciated that other shapes are possible.

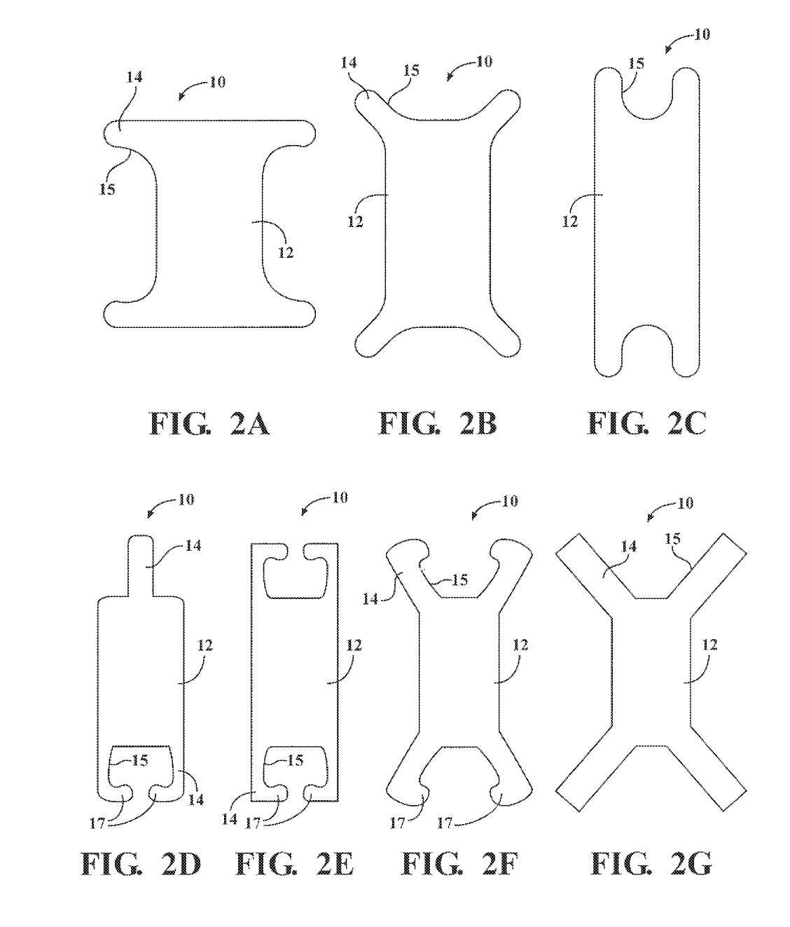

The at least two fingers 14 extend from opposite sides of the body 12, along a common plane with the flat configuration of the body 12, i.e., at least one finger 14 extends from each of the opposing sides of the body 12. In particular, because the flat configuration is defined due to the smaller dimension of the thickness of the body 12 as compared to length or width, the fingers 14 generally extend along a common plane with the length and/or width dimensions of the body, as generally shown in FIGS. 1 and 2. The fingers 14 provide anchoring points for tissue and facilitate implantation and allow explantation of the implantable device 10 without causing tissue damage. As described in further detail below in the context of the surgical technique for implanting the implantable devices 10, tissue (such as the iris of an eye as shown in FIG. 4) stretches over the fingers 14 after implantation. The tissue serves to secure the implantable device 10 in place by exerting compressive forces against the fingers 14 due to the stretched state of the tissue and a desire to return to a normal state.

To exploit the compressive forces of the tissue on the fingers 14, the implantable devices 10 may include the fingers 14 configured with various angles, tapers, and lengths, and the implantable devices 10 may be configured with different patterns of fingers 14 as depicted in FIGS. 1 and 2. In particular, referring to FIGS. 2A-G, the implantable device 10 may comprise a plurality of fingers 14 that extend from one side of the body 12, with at least one finger 14 extending from the opposite side of the body 12 in reference to the plurality of fingers 14. For example, in one embodiment, a plurality of fingers 14 can extend from one of the opposing sides of the body, with a single finger 14 extending from the opposing side of the body 12 from the plurality of fingers 14 as shown in FIG. 2D. Alternatively, as shown in FIGS. 2A-C and 2E-G, the implantable device 10 may comprise a plurality of fingers 14 that extend from both of the opposing sides of the body 12.

As shown in FIGS. 2B, 2F, and 2G, the fingers 14 may extend from the body 12 at an angle relative to each other. In any event, the fingers 14 may extend at various angles relative to the body 12 as extensively shown in the Figures. To these ends, when the implantable device 10 comprises a plurality of fingers 14 that extend from at least one side of the body 12, the fingers 14 define a gap 15 therebetween. The shape of the gap 15 is dependent upon the angle of the fingers 14 relative to the body 12, and may further be dependent upon other features of the fingers 14. For example, in one embodiment, the fingers 14 in the plurality of fingers 14 each have a protrusion 17 that extends toward another of the fingers 14 in the plurality of fingers 14, which protrusions 17 may assist with implantation of the implantable device 10 as described in further detail below. Typically, the protrusions 17 are disposed on a distal end of the respective fingers 14, spaced from the body, as shown in FIGS. 2D-F. As referred to herein, the distal end of the fingers 14 refers to the end of the finger 14 that is spaced from the body 12. In any event, the distal end of the fingers 14 is typically rounded to prevent the fingers 14 from cutting through the tissue in which the implantable devices 10 are embedded. In this regard, width and thickness of the fingers 14 may also be set to prevent the fingers 14 from cutting through the tissue, and the protrusions 17 may assist with prevention of cutting through the tissue.

The fingers 14 typically have a length on the micron scale, i.e., a length of less than 1 mm. In one embodiment, the fingers have a length of from about 100 μm to about 500 μm, alternatively from 250 μm to 500 μm. The fingers 14 typically have a width and thickness of from 100 μm to 500 μm.

The body 12 and the fingers 14 are typically integral and formed from the same material. Typically, the body 12 and fingers 14 are rigid and resist deformation, either plastic or elastic. In particular, the body 12 and fingers 14 are typically unbendable at room temperature, which enables the implantable device 10 to be implanted as described in detail below. To these ends, the body 12 and fingers 14 are typically formed from a ceramic material such as glass. However, it is to be appreciated that other materials, such as metals and polymers, may be used to form the body 12 and fingers 14. Further, it is to be appreciated that some bending of the body 12 and/or fingers 14 may be acceptable so long as the body 12 and fingers 14 do not bend when subjected to compressive forces as experienced when the implantable devices 10 are implanted in tissue. Because the implantable devices 10 are intended to be implanted into an organism, any materials used for the implantable devices 10 are preferably biocompatible and preferably will not damage the target tissue.

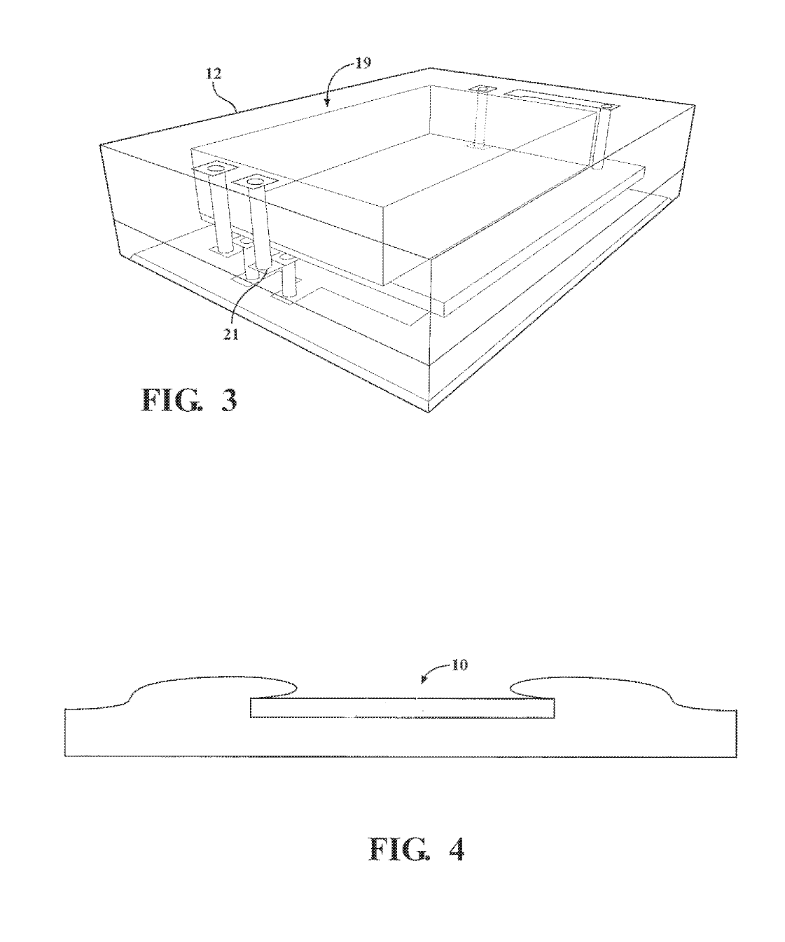

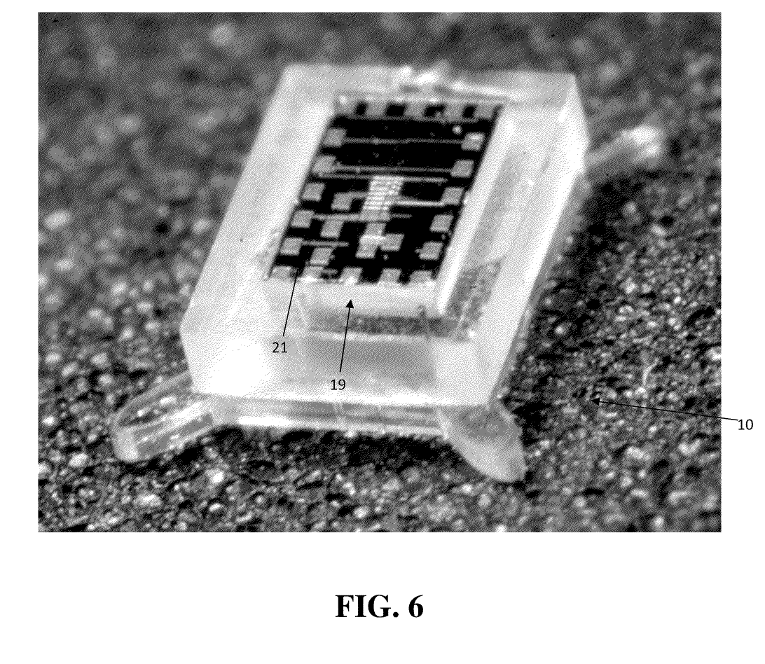

As set forth above, the implantable device comprises a diagnostic tool 19. The diagnostic tool 19 may be disposed on and/or embedded within the body 12 in the implantable device 10. The diagnostic tool 19 may be any tool that is capable of harvesting data once the implantable device 10 is implanted in tissue. The diagnostic tool 19 may be further defined as, but is not limited to, a pressure sensor, a pH sensor, or an electrical sensor. In one embodiment, to maintain a minimized profile of the diagnostic tool 19, the diagnostic tool 19 includes an integrated circuit 21. The integrated circuit 21 may be integrated directly into the body 12 of the implantable device 10, or may be formed in a separate substrate that is later bonded to the body 12 of the implantable device 10 (see, e.g., the device 10 shown in FIG. 6). FIG. 3 also shows an example of an integrated circuit 21.

A combined thickness of the body 12 and the diagnostic tool 19 is typically from 250 μm to 500 μm. However, it is to be appreciated that a thickness of the implantable device 10 may be larger depending upon the intended application and depending upon the type of diagnostic tool 19 that is included in the implantable device 10. In one embodiment, the overall dimensions of the implantable device 10, not including the fingers 14, may be 2 mm×1.5 mm×0.5 mm.

There is no particular limit to the manner in which the implantable device 10 is fabricated. In one embodiment, the implantable device 10 is fabricated through a glass-in-silicon reflow process is employed as described in U.S. Pre-Grant Publication No 2011/0091687, the entirety of which is hereby incorporated by reference, which enables formation of a variety of device shapes and features. A specific and detailed synopsis of one manner of fabricating the implantable device 10 is also provided in R.M. Hague et al., “A 3d Implantable Microsystem for Intraocular Pressure Monitoring Using a Glass-in-Silicon Reflow Process”, MEMS 2011, Cancun, MX, Jan 23-27, 2011, which is hereby incorporated by reference in its entirety.

The instant invention also includes a surgical technique to implant and fixate the implantable devices 10 into an organism. The surgical technique described herein has several advantages, including minimization of permanent damage to the tissue into which the implantable device 10 is implanted, easy and quick insertability, and simplified explantation (also without damaging tissue) if the implantable device 10 needs to be removed. Further, surgeries using implantable devices 10 of the scale shown in FIG. 1 may be considered minimally invasive, causing less damage to the tissue and therefore healing faster as well, often not requiring sutures. However, it is to be appreciated that the technique of the instant invention is not limited to a minimally invasive approach. Typically, the device 10 does not materially affect function of the tissue into which the device 10 is implanted. The main drawback of a minimally invasive approach is the limit it places on maximum physical dimensions of the implantable device 10.

For the surgical technique, the implantable device is positioned above the flexible and elastic tissue without penetrating the flexible and elastic tissue. Depending upon the particular application for the implantable devices 10, an incision may be made to provide access to tissue into which the implantable device 10 is to be implanted. Given the dimensions of the implantable device that are set forth above, an incision of 3 mm or less may be effective for enabling implantation of the implantable devices 10. For an incision of 3 mm or less, the width of the device is typically limited to 1.5 mm, allowing additional room for an insertion tool used to insert and implant the device through the incision. As one example, the surgical technique may be employed to implant the implantable devices 10 in the iris within the anterior chamber of the eye. In this embodiment, an incision may be made in the cornea to grant access to the iris (similar to more common procedures such as cataract surgery). The incision should be long enough to fit the width of the implantable device plus forceps or other implantation tool that used to hold the implantable device 10. When implanted in an iris of an eye, a small incision of 3 mm or less allows the cornea of the eye to self-heal without sutures, drastically reducing possible complications for the patient. For the surgical technique performed in the eye, a viscoelastic substance may be inserted into the anterior chamber of the eye to protect the corneal endothelium and maintain anterior chamber depth. The device, held by the forceps, is then inserted through the incision into the anterior chamber of the eye and positioned above the iris.

Once the tissue in which the implantable device 10 is to be implanted is accessible, the implantable device 10 can be manipulated to slide the finger(s) 14 on one side of the body 12 under a portion of the flexible and elastic tissue without penetrating the flexible and elastic tissue. The design of the fingers 14 concentrates application forces at the terminal ends thereof to enable the fingers to stretch and bunch up the tissue. As a result, the bunched tissue envelops the fingers 14 and, optionally, a portion of the body 12 of the implantable device 10. The other side of the implantable device is similarly manipulated to bunch up the tissue. In particular, the finger(s) on the opposing side of the body 12 are slid under another portion of the flexible and elastic tissue without penetrating the flexible and elastic tissue. The bunched tissue exerts force perpendicular to the body 12 and fingers 14 to fixate the device 10. In particular, the bunched tissue on either side of the implantable device 10 exerts force toward each other to hold the implantable device 10 in place. Since the fingers 14 are disposed on opposite sides of the body 12, the implantable device 10 is held in place by opposing forces from the bunched tissue on the respective sides of the implantable device 10. FIG. 4 illustrates the implantable device 10 after implantation into the tissue.

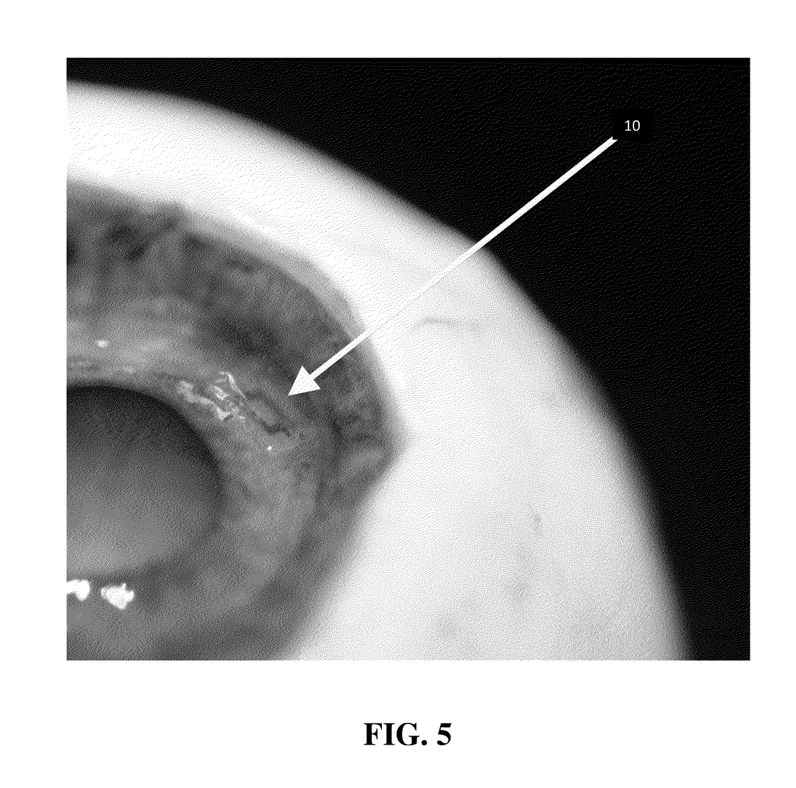

For the specific example in which the surgical technique is performed in the eye, with the implantable device 10 to be implanted in the iris, the implantable device is positioned above the iris at a target location. With gentle pressure, holding the implantable device 10 with forceps, the fingers 14 on one side of the implantable device 10 are seated into the iris, just below but generally parallel to the surface of the iris, with the fingers(s) on one side of the device angled toward the iris. Mostly muscle, the iris is very flexible and elastic so it stretches out, pushed by the rounded edges of the finger(s). This action causes the iris tissue to gather at the terminal ends of the fingers and begin to fold over as the device is pushed further into the iris stroma parallel to and near the iris surface. Once one end is submerged and a hillock is formed over the finger(s) 14, the other end of the device 10 is brought down and pushed into the surface of the iris. Due to the elasticity of the iris, the device 10 will be naturally pushed towards the opposite edge of the die, holding it in place by a compressive force generated by balancing the two ends of the device. The forceps may then be used to repeat the maneuver with the fingers 14 on the opposite side of the implantable device 10 to push the fingers 14 into the iris stroma. The implantable device 10 can be released once both sides of the implantable device 10 have portions of the iris folded over the distal ends of the fingers 14, maintaining a balance of forces that prevents the implantable device 10 from moving. The forceps may then be removed, followed by removal of the viscoelastic substance. The anterior chamber may be deepened with balanced salt solution. No sutures are required as the incision is self-sealing. The incision may be hydrated with balanced salt solution to assist in the self-sealing. FIG. 5 shows the successful implantation of a device 10 in the iris of a cadaver eye, although the cornea was removed to enable the device 10 to be seen. In order to explant the device, the implantation steps described above may simply be reversed. Although one advantage with the technique of the instant invention is the potential to avoid the need for sutures, an equally important advantage is the ability to remove the device 10, if necessary, without damaging any tissue.

Although the specific example provided above illustrates the surgical technique of the instant invention performed in the eye, and specifically in the iris of the eye, it is to be appreciated that the surgical technique and implantable device design are not exclusive to the iris or the ocular tissues; in fact, the surgical technique and implantable device 10 have much broader appeal for implantable medical devices in general. Successful implantation and explantation may depend on the mechanical properties of the tissue, precluding its use from certain areas of the body such as bone (non-flexible), though muscle or cartilage near bone may be a good candidate.

Obviously, many modifications and variations of the present invention are possible in light of the above teachings, and the invention may be practiced otherwise than as specifically described within the scope of the appended claims. It is to be understood that the appended claims are not limited to express and particular compounds, compositions, or methods described in the detailed description, which may vary between particular embodiments which fall within the scope of the appended claims. With respect to any Markush groups relied upon herein for describing particular features or aspects of various embodiments, it is to be appreciated that different, special, and/or unexpected results may be obtained from each member of the respective Markush group independent from all other Markush members. Each member of a Markush group may be relied upon individually and or in combination and provides adequate support for specific embodiments within the scope of the appended claims.

It is also to be understood that any ranges and subranges relied upon in describing various embodiments of the present invention independently and collectively fall within the scope of the appended claims, and are understood to describe and contemplate all ranges including whole and/or fractional values therein, even if such values are not expressly written herein. One of skill in the art readily recognizes that the enumerated ranges and subranges sufficiently describe and enable various embodiments of the present invention, and such ranges and subranges may be further delineated into relevant halves, thirds, quarters, fifths, and so on. As just one example, a range “of from 0.1 to 0.9” may be further delineated into a lower third, i.e., from 0.1 to 0.3, a middle third, i.e., from 0.4 to 0.6, and an upper third, i.e., from 0.7 to 0.9, which individually and collectively are within the scope of the appended claims, and may be relied upon individually and/or collectively and provide adequate support for specific embodiments within the scope of the appended claims. In addition, with respect to the language which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Finally, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

1. An implantable device that can be fixated to flexible and elastic tissue without sutures, said device comprising:
a body that presents a generally flat configuration;
at least two fingers extending from opposite sides of the body along a common plane with the flat configuration of the body; and
a diagnostic tool.

2. An implantable device as set forth in claim 1 wherein said body and fingers are integral and formed from the same material.

3. An implantable device as set forth in claim 1 wherein said body and fingers are formed from a ceramic or glass material.

4. An implantable device as set forth in claim 1 wherein said body and fingers are rigid and resist deformation.

5. An implantable device as set forth in claim 1 wherein a plurality of fingers extend from at least one side of the body, with at least one finger extending from an opposite side of the body in reference to the plurality of fingers.

6. An implantable device as set forth in claim 5 wherein a gap is defined between the plurality of fingers that extend from at least one side of the body.

7. An implantable device as set forth in claim 6 wherein the fingers in the plurality of fingers each have a protrusion that extends toward another of the fingers in the plurality of fingers.

8. An implantable device as set forth in claim 7 wherein the protrusions are disposed on a distal end of the respective fingers, spaced from the body.

9. An implantable device as set forth in claim 5 wherein the fingers in the plurality of fingers extend from the body at an angle relative to each other.

10. An implantable device as set forth in claim 1 wherein said diagnostic tool is further defined as a pressure sensor, pH sensor, or electrical sensor.

11. An implantable device as set forth in claim 1 wherein said diagnostic tool comprises an integrated circuit.

12. An implantable device as set forth in claim 1 wherein said body presents a length and width on the millimeter scale and wherein said fingers have a length on the micron scale.

13. A surgical technique for implanting an implantable device that can be fixated to flexible and elastic tissue without sutures, said technique comprising the steps of:
providing a device comprising a body that presents a generally flat configuration, at least two fingers extending from opposite sides of the body along a common plane with the flat configuration of the body, and a diagnostic tool;
positioning the implantable device above the flexible and elastic tissue at a target location;
sliding the finger on one side of the body under a portion of the flexible and elastic tissue without penetrating the flexible and elastic tissue; and
sliding the finger on the opposing side of the body under another portion of the flexible and elastic tissue without penetrating the flexible and elastic tissue;
wherein the flexible and elastic tissue exerts force perpendicular to the body and fingers to fixate the implantable device.

14. A technique as set forth in claim 13 wherein the flexible and elastic tissue to which the implantable device is fixated is further defined as the iris of an eye.

15. A technique as set forth in claim 14 wherein a viscoelastic substance is inserted into the anterior chamber of the eye prior to implantation of the implantable device in the iris.
